(19) United States
(12) Patent Application Publication
(10) Pub. No.: US 2007/0142837 A1
(43) Pub. Date: Jun. 21, 2007

(54) Threaded suture anchor with starting pitch

(76) Inventor: Peter J. Dreyfuss, Naples, FL (US)

Correspondence Address: DICKSTEIN SHAPIRO LLP, 1825 EYE STREET NW, Washington, DC, 20006-5403, US

(21) Appl. No.: 11/639,217

(22) Filed: Dec. 15, 2006

Related U.S. Application Data
(60) Provisional application No. 60/750,362, filed on Dec. 15, 2005.

(51) Int. Cl. A61B 17/58

(52) U.S. Cl. 606/73

(57) ABSTRACT

A suture anchor has a continuous thread spiralling around a tapering central core. At the distal end of the thread, the suture anchor has a starting pitch to facilitate insertion of the anchor into bone. The proximal end of the suture anchor receives at least one strand of suture. At the proximal end, the suture anchor has a large thread surface per turn of thread.

### CROSS-REFERENCE TO RELATED APPLICATIONS

This application claims the benefit of U.S. Provisional Application Ser. No. 60/750,362, filed Dec. 15, 2005, the entire disclosure of which is incorporated by reference herein.

### BACKGROUND OF THE INVENTION

1. Field of the Invention

The present invention relates to an apparatus and method for anchoring surgical suture to bone. More specifically, the present invention relates to a suture anchor having a thread with a starting pitch geometry configured to permit easier insertion into bone.

2. Description of the Related Art

When soft tissue tears away from bone, reattachment becomes necessary. Various devices, including sutures alone, screws, staples, wedges, and plugs have been used to secure soft tissue to bone. Recently, various types of threaded suture anchors have been developed for this purpose.

Suture anchors have been developed and designed to be inserted into a pre-drilled hole. Other suture anchors are self-tapping. Self-tapping screws are shown, for example, in U.S. Pat. No. 4,632,100, which discloses a cylindrical suture anchor. The suture anchor of U.S. Pat. No. 4,632,100 includes a drill bit at a leading end threads spaced from the drill bit for securing the anchor into the hole created by the drill bit. U.S. Pat. No. 5,370,662 discloses a self-tapping suture anchor having a flight of threads around a solid body. U.S. Pat. No. 5,156,616 discloses a similar suture anchor having an axial opening for holding a knotted piece of suture.

All of the above-noted suture anchors rely on a flight of threads disposed on the outer surface of a shank to secure the anchor to the bone. They all provide a relatively easy method of suture fixation in hard, cortical bone.

The above-described prior art suture anchors are difficult to secure to the bone, at least initially. For example, following an initial hammering of the suture anchor into the bone, excessive effort may be required to insert the suture anchor along the thread. Use of excessive force may increase the risk of injury to a patient from insertion of the suture anchor especially in softer bone sites, such as cancellous bone, or in bone tissue that has become compromised, such as in osteoporotic bone sites. The structure of cancellous bone is lattice-like, or spongy. Osteoporotic bone is the result of a condition that reduces the quantity of bone or atrophies skeletal tissue, causing a porous condition of the bones. These bone types may present a limited range of specific fixation points that are available to the surgeon.

Accordingly, a need exists for a suture anchor that can be started and secured easily and effectively, especially in softer types of bone.

### BRIEF SUMMARY OF THE INVENTION

The suture anchor of the present invention overcomes disadvantages of the prior art, such as those noted above, by providing a suture anchor having a thread spiralling helically around a central body and having a configuration that facilitates insertion of the suture anchor into the bone by providing a gradual change from a starting pitch (i.e, a thread disposed along the longitudinal axis of the suture anchor at the distal end of the anchor) to a helical or spiral pitch around the central body. By providing the starting pitch at the distal end of the suture anchor, the suture anchor can be inserted more readily into the bone without the need for additional or excessive force.

The present invention also provides a suture anchor and driver assembly for driving the anchor into bone. Advantageously, the suture anchor of the present invention is provided with threads formed in the shape of a corkscrew, to provide an increased percentage of thread surface area for each turn of the screw, as compared with known suture anchors, thus providing increased pull-out strength, and a decreased tendency for back-out. The driver for driving the corkscrew anchor may be provided with a cleat on the side of the handle. Suture threaded through the cannulated driver can be wrapped around the cleat and fixed for shipping in a slot in the cleat. The suture anchor and driver assembly can be shipped (pre-loaded with suture) as a sterile, surgery-ready unit.

Other features and advantages of the present invention will become apparent from the following description of the invention, which refers to the accompanying drawings.

### BRIEF DESCRIPTION OF THE DRAWINGS

FIG. 1 is a side view of a suture anchor according to an exemplary embodiment of the present invention.

FIG. 2 is a proximal end view of the suture anchor of FIG. 1.

FIG. 3 is a distal end view of the suture anchor of FIG. 1.

FIG. 4 is a side view of a suture anchor assembly including a suture anchor, threaded with suture, and a driver according to the present invention.

FIG. 5 illustrates a side view of a suture anchor according to another exemplary embodiment of the present invention.

FIG. 6 is a partial cross-sectional view of the suture anchor of FIG. 5, and illustrating a suture looped over a transverse anchor pin.

### DETAILED DESCRIPTION OF THE INVENTION

In the following detailed description, reference is made to various specific embodiments in which the invention may be practiced. These embodiments are described with sufficient detail to enable those skilled in the art to practice the invention, and it is to be understood that other embodiments may be employed, and that structural and logical changes may be made without departing from the spirit or scope of the present invention.

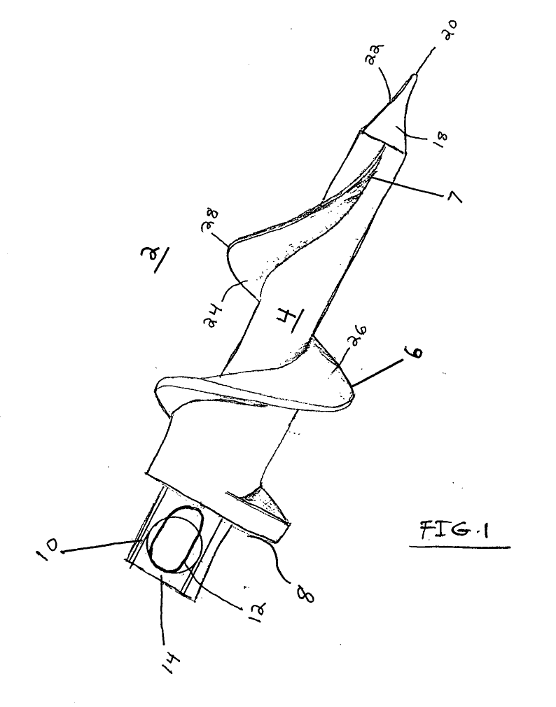

Referring now to the drawings, where like elements are designated by like reference numerals, FIGS. 1-6 illustrate exemplary embodiments of suture anchors of the present invention. As explained in detail below, the suture anchors of the present invention have a thread spiralling helically around a central body and includes a configuration that facilitates insertion of the suture anchor into the bone by providing a gradual change from a starting pitch (i.e, a thread disposed along the longitudinal axis of the suture anchor at the distal end of the anchor) to a helical or spiral pitch around the central body. By providing the starting pitch at the distal end of the suture anchor, the suture anchor can be inserted more readily into the bone without the need for additional or excessive force.

Advantageously, the threads of the suture anchor of the present invention are formed in the shape of a corkscrew, to provide an increased percentage of thread surface area for each turn of the screw, as compared with known suture anchors, thus providing increased pull-out strength, and a decreased tendency for back-out. The increase in the surface area of the thread is achieved in part by increasing the ratio of the outer diameter of the threads to the inner diameter of the threads. Preferably, the ratio is between 2.25 and 2.75. Most preferably, the ratio of the outer diameter to the inner diameter is 2.5.

Preferably, the suture anchor has a higher thread pitch than prior art screws, thus increasing the area of thread for each turn of the screw, which also leads to greater pull-out strength. Significantly, due to the increased pitch, fewer turns of the corkscrew screw thread are required to advance the suture anchor into position. Accordingly, the suture anchor is easy to install, and displaces less tissue material upon insertion than known suture anchors.

The present invention also provides a suture anchor and driver assembly for driving the corkscrew suture anchor into bone. The driver is formed of a cannulated tube secured to a cannulated handle. A socket (preferably hexagonal) formed on the distal end of the tube holds the suture anchor for rotation and installation into the bone. The outer diameter of the tube is about equal to or less than the outer diameter of the proximal end of the central body of the suture anchor.

The driver is optionally provided with a cleat on the side of the handle so that suture threaded through the cannulated driver can be wrapped around the cleat and fixed for shipping in a slot in the cleat using adhesive foam. One or more sutures, threaded through the suture anchor eye and up through the cannulated driver, can be pulled and secured around the cleat, the suture being pinched under tension. Advantageously, the tensioned suture helps to hold the suture anchor in place at the distal tip of the driver. The suture anchor and driver assembly can be shipped, pre-loaded with suture, as a sterile, surgery-ready unit.

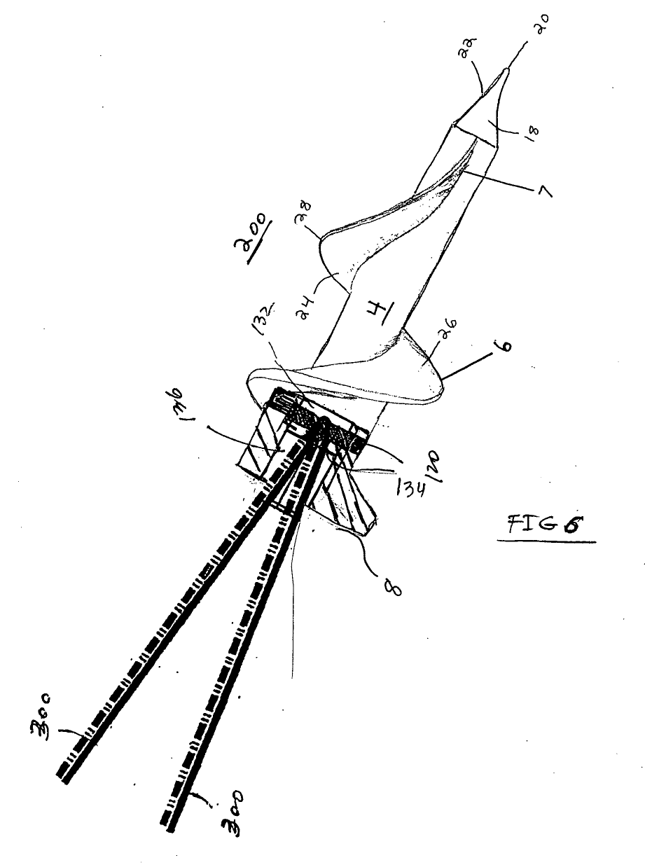

Referring now to the drawings, where like elements are designated by like reference numerals, FIGS. 1-3 illustrate suture anchor 2 according to a first exemplary embodiment of the present invention, and FIGS. 5 and 6 illustrate suture anchor 200 according to a second exemplary embodiment of the invention. As shown in FIGS. 1-3, the suture anchor 2 includes a body 4 provided in the shape of a tapered cylinder. A continuous thread 6 wraps around body 4 in a clockwise direction in the shape of a corkscrew, as shown. Advantageously, the thread 6 includes a configuration that facilitates insertion of the suture anchor into the bone by providing a gradual change from a starting pitch 7 (i.e, a thread disposed along the longitudinal axis of the suture anchor at the distal end of the anchor) to a helical or spiral pitch around the central body. By providing the starting pitch 7 at the distal end of the suture anchor 2, the suture anchor 2 can be inserted more readily into the bone without the need for additional or excessive force.

The anchor body 4 is preferably formed of a biocompatible material such as stainless steel or titanium alloy. The anchor body 4 is preferably circular in cross-section, and tapers from a maximum diameter near proximal end 8 to a minimum diameter toward distal end 14. Alternatively, the anchor body 4 may be formed of a translucent or transparent polymer material, and/or made of bioabsorbable materials such as polyglycolic or polylactic acid polymers. As used in the present application, the term “bioabsorbable” is considered to be interchangeable with the term “biodegradable,” “resorbable,” and “absorbable” to mean that the device can be absorbed by the body over time. Also, the measurements, angles and ratios between the dimensions of the suture anchor may be varied from those described above so as to be suitable for the conditions and applications in which the suture anchor is to be used.

As shown in FIG. 1, suture anchor 2 of the present invention may be provided at proximal end 8 of body 4 with drive head 10 (preferably hexagonal) having a suture eye 12. According to this exemplary embodiment, the suture eye 12 may have various configurations and geometries. Preferably, suture eye 12 is in the form of an oval aperture in the drive head and holds at least one piece of suture, preferably braided suture. Although reference to drive head 10 of suture anchor 2 of FIGS. 1-3 is made in this application as to the “hex drive head,” the invention is not limited to this exemplary embodiment, and it must be understood that drive head 10 may have any configuration and geometry, depending on the particular application and also on the characteristics of the driver.

At the junction between the hex drive head 10 and the central body 4, the circumference of the central body 4 is advantageously larger than the outer circumferential dimension of the hex drive head 10. Accordingly, the enlarged body adjacent the hex drive head forms a hole sufficient to accommodate a hex driver disposed over the hex drive head. This allows the suture anchor 2 to be at least partially countersunk below the surface of the bone upon installation, by preventing impingement of the distal end of the hex driver on the bone surface.

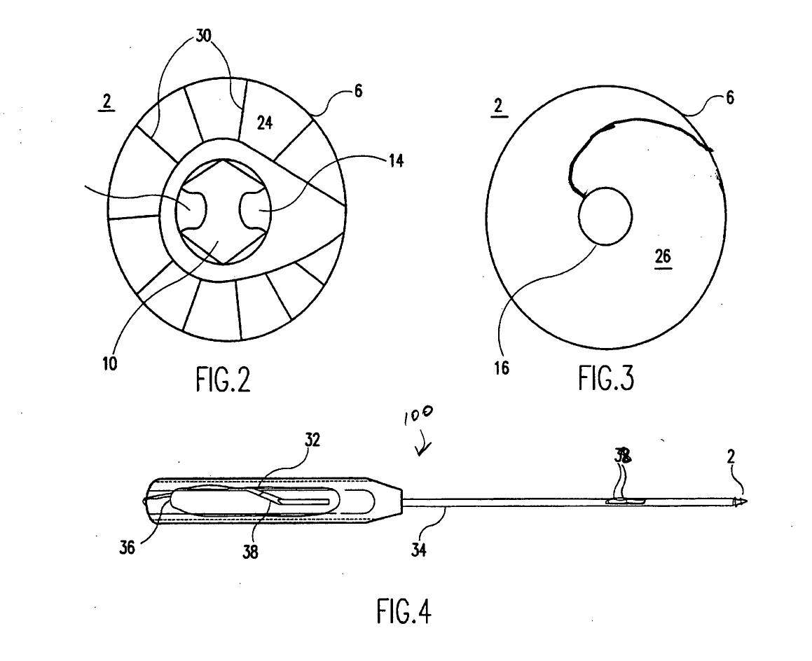

Channels 14 (FIG. 2) are formed along either side of the drive head 10 to accommodate the suture, for example, when the suture anchor is held in a suture anchor driver as described more fully below.

The central body 4 preferably tapers from the proximal end to terminate in a point 20 located at tip 18 of the distal end. The distal point 20 preferably is rounded to avoid possible breakage sometimes encountered when using a sharp point. As shown in FIG. 1, the rounded point 20 is approached by a concave, conically tapered surface 22. Conical taper 22 begins at the distal end of the screw thread, and features an angle of taper deeper than the taper of body 4.

Thread 6 illustrated in FIG. 1 has a proximal face 24, a distal face 26 and a break edge 28. Referring to FIG. 2, suture anchor 2 is shown from the proximal end. Radial ridge lines 30 are shown on proximal face 24 of corkscrew thread 6.

At the proximal end 8 of the anchor 2, the major (outside) diameter of the suture anchor thread of the present invention preferably is about 2.5 times the minor (inner) diameter of the thread, or the minor diameter of the body toward distal end 16. Accordingly, on a 5 mm diameter suture anchor, for example, where central core 4 is approximately 2 mm in diameter, the outer diameter of the thread is about 5 mm.

Preferably, between two and three flights or turns of thread 6 are provided along body 4, between proximal end 8 and distal end 16. The thickness of the thread increases proximally, such that at least the outer edge of the most proximal flight is thicker than the edge of each of the more distal flights, as shown in FIG. 1. Adjacent sections of each flight are separated by a gap that is determined by the number of turns per inch of the suture anchor thread. For example, a 5 mm suture anchor preferably has 6 turns per inch, while a 3.5 mm suture anchor preferably has about 9. Accordingly, on a 5 mm or 6.5 mm suture anchor, the pitch distance from flight to flight is about 0.167 inches. On a 3.5 mm suture anchor, pitch distance typically is about 0.118 inches.

The pull-out strength and minimal tissue damage are enhanced by the relatively compressed cross-sectional aspect of the thread 6, particularly in relation to the broad axial faces of the threads. The distal and proximal faces of the threads preferably form a square or rounded break edge at the outer diameter of the thread. In addition, and as noted above, the thickness of the thread increases proximally, such that at least the most proximal flight is thicker than each of the more distal flights. Increased back-out resistance may be enhanced by surface features, such as radial ridges 30 provided on the top and/or bottom faces of the screw threads. The surface features augment the engagement between the thread surfaces and the surrounding tissue once the suture anchor is installed.

FIGS. 5 and 6 illustrate another exemplary embodiment of a suture anchor 200 of the present invention, which is similar to suture anchor 2 of FIG. 1 in that suture anchor 200 also has a thread 6 spiralling helically around a central body 4 and including a configuration that facilitates insertion of the suture anchor into the bone by providing a gradual change from a starting pitch 7 (i.e, a thread disposed along the longitudinal axis of the suture anchor at the distal end of the anchor) to a helical or spiral pitch around the central body 4. However, suture anchor 200 differs from suture anchor 2 of FIG. 1 in that the suture anchor 200 is fully threaded and is not provided with a head drive 10 and suture eye 12. Rather, suture anchor 200 is provided at the proximal end 8 of the anchor with a hexagonally shaped bore 132 having an opening 120 at the proximal end of anchor body 4 and extending into the anchor body (e.g., by approximately one-fourth of the length thereof).

As shown in FIG. 6, cylindrical bore 136 having a diameter corresponding to that of the hexagonally shaped bore 132 extends from the distal end of the hexagonally shaped bore 132 to a position along the length of anchor body 4. The transition between hexagonally shaped bore 132 and cylindrical bore 136 forms an annular shoulder 134, against which the distal end of a hex driver abuts when inserted into the hexagonally shaped bore 132 to drive the anchor into bone.

Two longitudinal, diametrically opposite apertures 118 (FIG. 5) are formed in anchor body 4, the apertures 118 supporting a metal transverse anchor pin 120 which extends across cylindrical bore 136. As shown in FIG. 5, apertures 118 extend through and interrupt the threads 116 around anchor body 4 at approximately one-third of the length of the anchor body from the distal end thereof. One or more sutures 300 are secured to the anchor by looping the suture(s) around anchor pin 120, as shown in FIG. 6. Although the anchor pin 120 is illustrated in FIG. 6 as oriented transversal to the longitudinal axis of the anchor body 4, the anchor pin 120 may form any angle with the longitudinal axis and, thus, the invention is not limited to anchor pin 120 forming an angle of about ninety degrees with the longitudinal axis of the anchor body 4.

The present invention also provides a method of anchoring suture in bone using the suture anchor 2, 200 of the present invention. The method includes the steps of: (i) threading suture through the suture eye or around the pin at the proximal end of the suture anchor 2, 200 described above with reference to FIGS. 1-6; (ii) coupling a driver with the suture anchor 2, 200; and turning the driver (with the suture anchor 2, 200) to advance the suture anchor 2, 200 into the bone.

The anchors of the present invention may be used for various arthroscopic procedures. Advantageously, the suture anchor can be installed using a hollow, cannulated grasper as described in U.S. Pat. No. 5,466,243 to Schmieding, the disclosure of which is incorporated herein by reference. The anchors are also advantageous for open and mini-open surgical procedures, such as open rotator cuff repair, as described in U.S. Pat. No. 5,575,801 to Habermeyer et al.

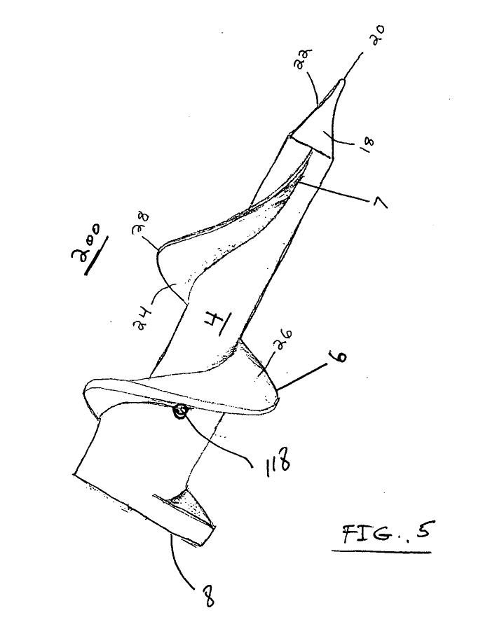

FIG. 4 illustrates an exemplary suture anchor/driver assembly 100 including suture anchor 2 of FIGS. 1-3 threaded with two pieces of suture 32 and held on a cannulated suture anchor driver 34. Optionally, the suture anchor can be distributed with at least one strand of suture already threaded through the eye 12. For example, FIG. 4 illustrates suture strands 32 attached to the eye 12 and threaded through the cannula of driver 34, allowing the sutures to slide smoothly with minimal friction. In an exemplary embodiment, the suture strands 32 may be FiberWire composite sutures of alternating colors to maximize repair strength, aid in suture management and provide superior tying characteristics.

Suture strands 32 are threaded through the cannula of the driver 34 and secured on a hook and cleat 36 (FIG. 4) on the handle of the driver, to allow the distal end of the head of the driver to be inserted into the opening of the anchor 2 so that the suture anchor is driven into a pilot hole.

As shown in FIG. 4, the suture 32 is wrapped around cleat 36. Tension on the suture aids in retaining the suture anchor in the distal end of the driver. The suture may be held in place in slot 38 using a foam adhesive, for example, during shipping. Assembly 100 may be provided as a sterile unit ready for surgical application.

Sutures anchors according to the present invention can be used for arthroscopic procedures. The anchors are also advantageous for open and mini-open surgical procedures. Specific examples of applicable procedures include cortical bone-soft tissue fixation, Bankart and SLAP shoulder repairs.

A surgical method employing a threaded suture anchor with starting pitch, such as the threaded suture anchor 2 of FIGS. 1-4 or fully threaded suture anchor 200 of FIGS. 5-6, generally includes pre-forming a hole for insertion of the anchor using a punch and a tap. The anchor is then engaged with a cannulated driver, such as driver 34 of FIG. 4, inserted into the pilot hole and turned to advance the suture anchor into the bone. The driver is then removed, and the suture extending through the internal suture eye 12, or around transversal pin 120, can then be manipulated and tied to secure soft tissue to bone.

Although the present invention has been described in relation to particular embodiments thereof, many other variations and modifications and other uses will become apparent to those skilled in the art. Therefore, the present invention is to be limited not by the specific disclosure herein, but only by the appended claims.

What is claimed is:

1. A suture anchor for securing suture to soft bone, comprising:
a central body having a central axis, a distal end and a proximal end; and
a continuous thread for anchoring the suture anchor in soft bone, the continuous thread being disposed in a spiral around the central body and including a starting pitch at the distal end of the central body, wherein at least a portion of the thread is oriented longitudinally relative to the central axis of the suture anchor to facilitate insertion of the anchor.

2. The suture anchor of claim 1, wherein the thread has an inner diameter, an outer diameter and thickness, wherein the thickness of the thread at the outer edge of the thread increases proximally along a portion of the thread.

3. The suture anchor of claim 2, wherein the outer diameter of the thread is at least twice the inner diameter of the thread at the proximal end of the central body.

4. The suture anchor of claim 1, further comprising a tip disposed at the distal end of the central body.

5. The suture anchor of claim 4, wherein the tip has a tip taper which is greater than a taper of the central body.

6. The suture anchor of claim 1 further comprising a drive head disposed on the proximal end of the central body, the drive head including an eyelet for receiving at least one strand of flexible material.

7. The suture anchor of claim 6, wherein the strand of flexible material is suture.

8. The suture anchor of claim 6, further comprising suture channels located on the drive head.

9. The suture anchor of claim 6, wherein the drive head is hexagonal.

10. The suture anchor of claim 1 further comprising a central bore extending from an opening at the proximal end of the anchor body through a portion of the length thereof, and a member disposed transversely in the central bore and having ends that extend across the bore to the anchor body.

11. The suture anchor of claim 2, wherein the ratio of the outer diameter to the inner diameter of the thread is between about 2.25 and about 2.75 along a portion of the thread.

12. A method of anchoring suture in bone, the method comprising the steps:
providing a suture anchor comprising a central body having a central axis, a distal end and a proximal end, the central body being tapered along a major portion thereof from a maximum diameter near the proximal end to a minimum diameter toward the distal end; and a continuous thread for anchoring the suture anchor in soft bone, the continuous thread being disposed in a spiral around the central body and including a starting pitch at the distal end of the central body, wherein at least a portion of the thread is oriented longitudinally relative to the central axis of the suture anchor to facilitate insertion of the anchor;
threading suture through the proximal end of the suture anchor; and
driving the suture anchor into the bone.

13. The method of claim 12, wherein the bone is in the shoulder.

14. The method of claim 12, wherein the anchor is used for rotator cuff repair.

15. The method of claim 12, wherein the bone is soft or cancellous bone.

16. The method of claim 12, wherein the suture anchor is installed arthroscopically.

17. The method of claim 12, wherein the suture anchor further comprises a drive head disposed on the proximal end of the central body, the drive head including an eyelet for receiving at least one strand of flexible material.

18. The method of claim 17 further comprising the step of threading suture through the eyelet.

19. The method of claim 12, wherein the suture anchor further comprises a central bore extending from an opening at the proximal end of the anchor body through a portion of the length thereof, and a member disposed transversely in the central bore and having ends that extend across the bore to the anchor body.

20. The method of claim 19 further comprising the step of threading suture around the member disposed transversely in the central bore.

21. An assembly of a driver and suture anchor for securing suture to soft bone, comprising:
a driver for driving a suture anchor into soft bone, the driver comprising a tubular shaft having a distal end and a proximal end, and a handle disposed on the proximal end of the tubular shaft; and
a suture anchor inserted into the distal end of the driver, the suture anchor comprising a central body having a central axis, a distal end and a proximal end, the central body being tapered along a major portion thereof from a maximum diameter near the proximal end to a minimum diameter toward the distal end; a drive head disposed on the proximal end of the central body, the drive head being configured to receive at least one strand of flexible material; and a continuous thread for anchoring the suture anchor in soft bone, the continuous thread being disposed in a spiral around the central body and including a starting pitch at the distal end of the central body, wherein at least a portion of the thread is oriented longitudinally relative to the central axis of the suture anchor to facilitate insertion of the anchor.

22. The suture anchor and driver assembly of claim 21, further comprising a cleat for holding the suture on an outer surface of the handle of the driver.

23. The suture anchor and driver assembly of claim 22, wherein the cleat includes a slot for holding suture.
